(19) United States
(12) Patent Application Publication
(10) Pub. No.: US 2026/0053768 A1
(43) Pub. Date: Feb. 26, 2026

(54) Sustained Nitric Oxide Releasing Drug Delivery Systems

(71) Applicant: Maher Hadaya, Hamilton, NJ (US)

(72) Inventor: Maher Hadaya, Hamilton, NJ (US)

(21) Appl. No.: 19/198,094

(22) Filed: May 4, 2025

Related U.S. Application Data
(60) Provisional application No. 63/643,479, filed on May 7, 2024.

(51) Int. Cl. A61K 31/215; A61K 9/06; A61K 9/1273; A61K 9/1277; A61P 27/02

(52) U.S. Cl. CPC A61K 31/215; A61K 9/06; A61K 9/1273; A61K 9/1277; A61P 27/02

(57) ABSTRACT

Disclosed herein are drug delivery vehicle compositions, comprising lipid nanoparticle-forming molecules, a nitric oxide donor (NO-donor) molecule, and a hydrogel. Also disclosed are methods of making the same by mixing the lipid nanoparticle-forming molecules and the NO-donor in a hydrophobic solvent to form a first mixture; and adding a mixture of hydrogel and water, and optionally a buffer solution, to the first mixture to form the drug delivery vehicle composition. In addition, disclosed herein are methods of treating a patient suffering from a disease that can be treated with nitric oxide, the method comprising identifying a patient in need thereof, and administering to the patient a drug delivery vehicle disclosed herein.

### RELATED APPLICATIONS

This application claims priority to U.S. provisional application No. 63/643,479, filed May 7, 2024, and entitled “Sustained Nitric Oxide Releasing, Chitosan Coated, Extended-Release Drug Delivery System”, which is incorporated herein by reference as if set forth in its entirety.

### FIELD OF THE INVENTION

The present invention is in the field of time release delivery of nitric oxide (“NO”) for a targeted drug delivery using lipid nanoparticle vehicles.

### BACKGROUND OF THE DISCLOSURE

Micelles have been researched for use in delivering drugs for many years. They can increase the concentration of drugs at target sites due to the enhanced permeability and retention effect. However, their effectiveness is reduced by instability in the body, often causing them to break apart and release drugs too early. This premature release can lead to high toxicity and ineffective treatment. Efforts have focused on increasing micelle stability by lowering the critical micelle forming concentration and extending circulation time in the blood.

A major issue with micelles is their tendency to split apart when the concentration drops below a certain level, especially after injection. This breakdown can lead to a quick release of the drugs into the bloodstream, diminishing the benefits of optimized drug carriers and resulting in poor distribution and therapeutic effects. For instance, the drug Taxotere® is quickly eliminated from circulation after injection, hindering its therapeutic effectiveness. This presents a significant challenge in developing stable micelles for drug delivery.

Nitric oxide (NO) is a small gas that helps regulate the cardiovascular system by causing blood vessels to widen. It has various functions, including reducing inflammation and preventing blood clotting. NO is produced in the body through an enzyme called nitric oxide synthase. Delivering NO in the body is challenging due to its instability and rapid decay, limiting its therapeutic use. Various NO donors have been developed to release NO in a controlled manner, including compounds like NONOate.

NO has also shown potential in treating conditions such as glaucoma by lowering intraocular pressure and promoting healing in the cornea. However, it is crucial to control the amount and timing of NO release, as excessive amounts can cause toxicity. Current therapies for using NO are limited and often fail to exploit its full potential. The goal is to develop personalized NO delivery systems that can adjust dosing and release based on individual patient needs.

Current nitric oxide delivery systems face problems, including limited sustained release, scalability, and personalization challenges. Many existing methods tie the release of NO to drug delivery, making it difficult to control the dosage of NO independently. In addition, various systems present significant issues like toxicity, scalability problems, and complex manufacturing, which do not support effective personalized treatments. There is a clear gap in the ability to deliver nitric oxide in a manner that is both effective and adaptable to individual patient needs.

### SUMMARY OF THE INVENTION

Disclosed herein are drug delivery vehicle compositions, comprising a lipid nanoparticle-forming molecules, a nitric oxide donor (NO-donor) molecule, and a hydrogel. Also disclosed are methods of making the same by mixing the lipid nanoparticle-forming molecules and the NO-donor in a hydrophobic solvent to form a first mixture; and adding a mixture of hydrogel and water, and optionally a buffer solution, to the first mixture to form the drug delivery vehicle composition. In addition, disclosed herein are methods of treating a patient suffering from a disease that can be treated with nitric oxide, the method comprising identifying a patient in need thereof, and administering to the patient a drug delivery vehicle disclosed herein.

### DETAILED DESCRIPTION OF THE EMBODIMENTS

The present disclosure is generally directed to drug delivery vehicles for the delivery of nitric oxide (NO) or other therapeutic agents. Briefly, a nitric oxide donor molecule is trapped within a lipid nanoparticle and the lipid nanoparticle is in turn trapped within a hydrogel. The hydrogel can be applied to an area of a patient's body that can benefit from NO treatment. The NO within the lipid nanoparticle leaches out in a time release manner and the hydrogel keeps the therapeutic agent fixed in place and provides for the slow release of NO.

Lipids are a broad group of organic compounds which include fats, waxes, sterols, fat-soluble vitamins (such as vitamins A, D, E and K), monoglycerides, diglycerides, phospholipids, and others. Some lipids are amphiphilic small molecules; the amphiphilic nature thereof allows them to form nanoparticles, such as vesicles, micelles, microemulsions, multilamellar/unilamellar liposomes, or membranes in an aqueous environment. The lipid nanoparticles, i.e., nanoparticles made from lipids, provide a homogeneous biphasic environment, by way of which molecules having a different hydrophilicity than the solvent can be solubilized. The amphiphilic lipid nanoparticles have a hydrophilic head and a hydrophobic tail. In a hydrophobic environment, they arrange themselves such that the hydrophilic head is inside the nanoparticle and the hydrophobic tail forms the outer shell. Conversely, in a hydrophilic environment, the lipid nanoparticles arrange themselves such that the hydrophobic tail is inside the nanoparticle and the hydrophilic head forms the outer shell. Thus, in a hydrophobic environment, the lipid nanoparticles can solubilize a hydrophilic molecule by encasing it in the hydrophilic interior of the nanoparticle. Likewise, in a hydrophilic environment, the lipid nanoparticles can solubilize a hydrophobic molecule by encasing it in the hydrophobic interior of the nanoparticle. The lipid nanoparticles can do so either as free floating nanoparticles or as aggregates in colloidal form.

Unless otherwise specified, or clear from the context, the words “lipid nanoparticle,” “micelle,” “microemulsion,” and “vesicle” are used interchangeably throughout the present disclosure.

Micelles are a well-known drug delivery vehicle, which generally are formed as a colloidal suspension in a solvent. More particularly, a micelle typically has a hydrophilic shell that surrounds a hydrophobic core such that a desired therapeutic drug can be held within the core until injected into a patient. Microemulsions are similar to micelles, but with significant differences. While they are both generally composed of surfactant, co-surfactant, and lipid, the key difference in composition is the amount of lipid. The surfactant-to-lipid ratio is higher in a micelle than in a microemulsion. There are associated stability differences between microemulsions and micelles as well. Under physiological conditions, micelles degrade rapidly upon dilution to below Critical Micellar Concentration (CMC), while a microemulsion degrades overtime and is kinetically stable in the short term.

Generally, the surfactant and cosurfactant constituents of an amphiphilic lipid nanoparticle have a polar hydrophilic head, and a nonpolar hydrophobic tail. In an aqueous environment, the constituents of a lipid nanoparticle self-organize into lipid nanoparticles without energy input, because the lipid nanoparticle forms the most thermodynamically stable structure where only the hydrophilic ends are exposed to the water. This spontaneous self-organization allows lipid nanoparticles to be highly adaptable to different aqueous environments and be easily scalable.

As is recognized in the art, nitric oxide (NO) treatment is understood to have enormous potential, but current approved drug delivery vehicles cannot deliver a customizable dose of NO. The customization may involve the particular dose of NO for a particular treatment (for example, the regeneration of the cornea requires a much lower dose of NO than the treatment of intraocular pressure (IOP)), or the duration the dose is applied or that NO is allowed to take effect, or a particular dose needed due to the particular idiosyncrasies of a particular patient. Other drug delivery vehicles that have been developed for sustained NO release are not easily adaptable for personalized medicine.

The drug delivery vehicles disclosed herein overcome these deficiencies by enabling target delivery of a stable, controlled time release, therapeutically effective quantity of NO to a target site in a patient, wherein the drug delivery vehicles are scalable and modifiable for delivery of NO at a personalized level. The presently disclosed drug delivery vehicles allow easy integration of NO treatment into an already existing treatment regimen or can be applied simultaneously with other drugs or other treatments, targeting multiple conditions at once. The use of the presently disclosed drug delivery vehicles allows clinicians to more easily avail themselves of the synergistic use of different treatments by making the application of NO relatively simple and targeted.

Thus, in one aspect, disclosed herein are compositions of lipid nanoparticles developed for the delivery of nitric oxide (NO) as a therapeutic agent in a patient in need of such treatment. The compositions disclosed herein are also referred to as “drug delivery vehicles.”

Throughout the present disclosure and the claims, the word “inert” means a compound or composition that is physiologically acceptable and under physiological conditions does not cause intolerable adverse events, such as itching, rash, dizziness, anaphylaxis, and the like. An “inert” compound may undergo reactions under physiological conditions (such as oxidation, reduction, hydrolyzation, condensation, and the like) but the reactions do not significantly affect the intended function of the drug delivery vehicles, nor do the reactions cause adverse effects in the patient.

Throughout the present disclosure and the claims, the word “about” or “approximately” means a value within a range of ±25%, or a range of ±10%, or a range of ±5% of the stated value. Thus, for example, a value of “about 50” or “approximately 50” means a value in the range of 37.5-72.5, or in the range of 45-55, or in the range of 47.5-52.5.

In some embodiments, the drug delivery vehicles disclosed herein comprise the following component, or substituents: 1) an NO-donor, 2) lipid nanoparticle-forming molecules, i.e., those having a hydrophobic tail and a hydrophilic head, and 3) a hydrogel.

### Nitric Oxide Donors

Nitric oxide (NO) donors are pharmacologically active substances that release NO in vivo or in vitro. Generally, NO-donors are divided into direct donors, for example, nicorandil, the sydnonimines and sodium nitrite, and indirect donors, for example nitrovasodilators such as nitroglycerin (NTG), isosorbide mononitrate (ISMN) and isosorbide dinitrate (ISDN). Most NO-donors currently available are hydrophilic molecules.

In some embodiments, the inert NO-donor of the present compositions is prepared from a triterpenoid compound. In certain embodiments, the triterpenoid compound is a tricyclic triterpenoid compound, a tetracyclic triterpenoid compound, or a pentacyclic triterpenoid compound. In some embodiments, the triterpenoid compound is selected from the group consisting of ursolic acid, 23-hydroxy ursolic acid, oleanolic acid, uvaol, -amyrin, amyrin, asiatic acid, erythrodiol, hederagenin, madecassic acid, corosolic acid, betulin, and bardoxolone.

In some embodiments, ursolic acid is used as the basis for the inert NO-donor. This particular NO-donor, which is a hydrophobic molecule, is prepared in a two-step process.

In the first step, ursolic acid is mixed with a haloalkane in a polar solvent. In some embodiments, a mild base is also added to the reaction mixture. The mixture is stirred at a temperature of between about 20° C. to about 150° C. for a duration of between about 2 hours to about 12 hours.

In some embodiments, the haloalkane is a straight-chain alkane, whereas in other embodiments, the haloalkane is a branched alkane. In certain embodiments, the alkane is a C₂-C₁₅ alkane. By “C₂-C₁₅ alkane” it is meant that the alkane chain comprises between 2 to 15 carbon atoms. In other embodiments, the alkane is a C₂-C₁₀ alkane, whereas in still other embodiments, the alkane is a C₂-C₇ alkane. In some embodiments, the halogen of the haloalkane is selected from the group consisting of fluorine, chlorine, bromine, and iodine. In some embodiments, the haloalkane comprises two or more halogen atoms. In other embodiments, the haloalkane is a perhaloalkane. In some embodiments, the alkane is 1,5-dibromopentane.

In some embodiments, the polar solvent is a carbonyl-containing solvent or a nitrile. In certain of these embodiments, the solvent is an amide, an ester, a ketone, or an aldehyde. In some embodiments, each of the one or more alkyl chain(s) in the solvent is a C₁-C₆ alkyl, whereas in still other embodiments, the alkyl is a C₁-C₄ alkyl. In some embodiments, the solvent is dimethylformamide (DMF).

In some embodiments, the mild base is an organic base. Examples of organic acids include, but are not limited to, primary, secondary, or tertiary amines, carboxylates, alcohols, sterically hindered alkoxides, mercaptans, carbonate, bicarbonate, and the like. In other embodiments, the mild base is an inorganic base. Examples of inorganic bases include sulfate, bisulfate, nitrates, phosphates, and the like. In some embodiments, the mild base is potassium carbonate.

In some embodiments, the reaction mixture is stirred at a temperature of between about 10° C. to about 150° C., or at a temperature of between about 20° C. to about 100° C., or at a temperature of between about 30° C. to about 50° C., or at a temperature of about 35° C. In other embodiments, the reaction mixture is heated to reflux.

In some embodiments, the mixture is stirred for a duration of between about 2 hours to about 48 hours, or between about 5 hours to about 36 hours, or between about 8 hours to about 24 hours, or for about 12 hours.

In some embodiments, the reaction mixture for this first step comprises the following reactants/reagents in the following weight percents:

| | |
|---|---|
| DMF | 1-70% by weight |
| Ursolic Acid | 1-70% by weight |
| 1,5-Dibromopentane | 1-70% by weight |
| Potassium Carbonate | 1-70% by weight |

This first step of the reaction results in the intermediate Compound 1.

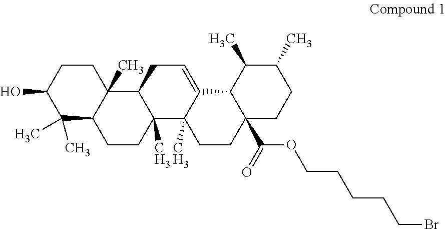 [SMILES: C[C@H]1[C@H](C)CC[C@]2(C(=O)OCCCCCBr)CC[C@]3(C)C(=CC[C@@H]4[C@@]5(C)CC[C@H](O)C(C)(C)[C@@H]5CC[C@]43C)[C@H]12]

In the second step, Compound 1 is reacted with silver nitrate in a polar solvent, as d escribed above. In some embodiments, the polar solvent is acetonitrile.

In some embodiments, the reaction mixture is stirred at a temperature of between about 20° C. to about 150° C., or at a temperature of between about 50° C. to about 120° C., or at a temperature of between about 70° C. to about 100° C. In some embodiments, the reaction mixture is stirred at a temperature of between about 80° C. to about 85° C. In other embodiments, the reaction mixture is heated to reflux.

In some embodiments, the mixture is stirred for a duration of between about 2 hours to about 100 hours, or between about 10 hours to about 80 hours, or between about 30 hours to about 50 hours. In some embodiments, the mixture is stirred for 48 hours.

At the conclusion of the reaction, the product, Compound 2, is purified using procedures well known in the art, such as column chromatography or thin-layer chromatography.

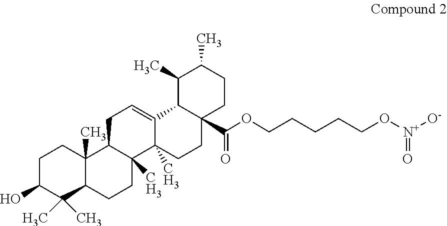 [SMILES: C[C@H]1[C@H](C)CC[C@]2(C(=O)OCCCCCO[N+](=O)[O-])CC[C@]3(C)C(=CC[C@@H]4[C@@]5(C)CC[C@H](O)C(C)(C)[C@@H]5CC[C@]43C)[C@H]12]

In some embodiments, the reaction mixture for this second step comprised the following reactants/reagents in the following weight percents:

| | |
|---|---|
| Acetonitrile | 1-70% by weight |
| Compound 1 | 1-70% by weight |
| Silver Nitrate | 1-70% by weight |

In one embodiment, the NO-donor was an organic nitrate ester. This group has been shown to release nitric oxide at a steady rate in response to exposure to esterases, is well-tolerated, and is known to be therapeutically efficacious.

In other embodiments, sunflower oil was used to make a nitrated lipid. The NO moiety was attached to the sunflower oil, using the well-known plasma acidified nitration process.

All NO-donors currently known or identified in the future are included within the scope of the present disclosure. In particular, an NO-donor most suited for the compositions and methods disclosed herein is inert and is sufficiently hydrophobic to integrate with lipid nanoparticles.

The NO-donor also preferably has a long shelf-life. For example, it undergoes oxidation very slowly or not at all, and/or it is insensitive to light. By “long shelf-life” it is meant that during the average time it takes for similar medications to be prepared, packaged, shipped, stored, and finally used, the extent of decomposition of the composition is within a range that does not affect its pharmacological efficacy to a significant degree.

Those of ordinary skill in the art recognize that the present drug delivery vehicles can also be used with those NO-donors that have short shelf-lives or are light sensitive. The present compositions entrap the NO-donor within the lipid nanoparticle cage, thereby isolating the donor molecule from other compounds, such as salts, solvents, and the like, that cause the decomposition of the unstable NO-donor.

### Lipid Nanoparticles

In another aspect, disclosed herein are methods of making the lipid nanoparticles or drug delivery vehicles disclosed herein. The following discussion is in the context of micelles for clarity. The ordinary artisan recognizes that this discussion is applicable to all the lipid nanoparticles within the scope of the present disclosure.

In a typical micelle, the core is hydrophobic while the shell is hydrophilic. Micelles can form in an inverted form, i.e., where the core is hydrophilic, and the shell is hydrophobic. The inverted micelle can be made to invert to form a typical micelle, and vice versa.

In some embodiments, the micelle is made up of lipids, having a hydrophilic head and a hydrophobic tail. In certain embodiments, the lipid is selected from, but is not limited to, free fatty acids, glycerolipids (e.g., mono-, di-, or triglycerides), glycerophospholipids, sphingolipids, saccharolipids, polyketides, sterol lipids (e.g., cholesterol), and prenol lipids. In some embodiments, the alkyl chain of the lipid comprises between about 5 to about 30 carbon atoms. In some embodiments, the lipid is TWEEN® 80 or a phospholipid.

In some embodiments, a surfactant is added to the lipid solution. Some examples of surfactants that are suitable for use in the compositions described herein include, but are not limited to, the following:
Anionic: Sodium linear alkylbenzene sulfonate (LABS); sodium lauryl sulfate; sodium lauryl ether sulfates; petroleum sulfonates; linosulfonates; naphthalene sulfonates, branched alkylbenzene sulfonates; linear alkylbenzene sulfonates; fatty acid alkylolamide sulfosuccinate; alcohol sulfates; dioctyl ester of sodium sulfosuccinic acid.
Cationic: Stearalkonium chloride; benzalkonium chloride; quaternary ammonium compounds; amine compounds; ethosulfate compounds.
Non-ionic: Dodecyl dimethylamine oxide; coco diethanol-amide alcohol ethoxylates; linear primary alcohol polyethoxylate; alkylphenol ethoxylates; alcohol ethoxylates; EO/PO polyol block polymers; polyethylene glycol esters; fatty acid alkanolamides.
Amphoteric: Cocoamphocarboxyglycinate; cocamidopropylbetaine; betaines; imidazolines.

In addition to those listed above, suitable nonionic surfactants include alkanolamides, amine oxides, block polymers, ethoxylated primary and secondary alcohols, ethoxylated alkylphenols, ethoxylated fatty esters, sorbitan derivatives, glycerol esters, propoxylated and ethoxylated fatty acids, alcohols, and alkyl phenols, alkyl glucoside glycol esters, polymeric polysaccharides, sulfates and sulfonates of ethoxylated alkylphenols, silicone glycol copolymers, polymeric surfactants, and Gemini surfactants that have two hydrophilic heads connected to two or three hydrophobic tails. Suitable anionic surfactants include ethoxylated amines and/or amides, sulfosuccinates and derivatives, sulfates of ethoxylated alcohols, sulfates of alcohols, sulfonates and sulfonic acid derivatives, phosphate esters, and polymeric surfactants. Suitable amphoteric surfactants include betaine derivatives. Suitable cationic surfactants include amine surfactants, quaternary ammonium chloride surfactants, ethyldimonium ethosulfates, and other quaternary surfactants. Those skilled in the art will recognize that other and further surfactants are potentially useful in the compositions depending on the particular application.

In some embodiments, the surfactant is an oleate-derived surfactant, such as a polyoxyethylene sorbitan monooleate (for example polyoxyethylene (80) sorbitan monooleate, known commercially as TWEEN® 80) or a laurate-derived surfactant, such as a polyoxyethylene sorbitan monolaurate (for example polyoxyethylene (20) sorbitan monolaurate, known commercially as TWEEN® 20).

In some embodiments, a co-surfactant is also added to the reaction mixture. The co-surfactant is also selected from the list of surfactants disclosed above, except that the co-surfactant is present at a lower concentration than the surfactant. In some embodiments, the ratio of surfactant to co-surfactant is between about 90:10 to about 60:40.

To the mixture of the lipid and surfactant the NO-donor added. Because a hydrophobic solvent is used at this stage, the compounds spontaneously interact to form reverse micelles. As stated above, the reverse micelle is a generally spherical structure, where the hydrophilic heads of the lipid and the surfactant, if present, form the core of the sphere and the hydrophobic lipid side chains form the shell. Thus, the hydrophobic side chains interact with the hydrophobic environment of the solvent, and the hydrophilic ends are protected in their own polar environment inside the micelle. In this arrangement, the NO-donor, which is highly hydrophobic, is positioned within the hydrophobic environment of the shell.

In some embodiments, the NO-donor is added to the emulsion stock solution, which is then added to the hydrogel solution prior to the inversion step.

### Micelle Inversion and Hydrogel Formation

By adding water or an aqueous buffer solution to the inverted micelle mixture described above, the dipole moment of the solution begins to increase. At some point, the solvent environment becomes sufficiently polar that the micelles invert to expose their hydrophilic heads to the solvent and bury the hydrophobic side chains inside the micelle. That is, the micelles invert to form a typical micelle. The NO-donor, which is hydrophobic and was interacting with the hydrophobic side chains, follows the hydrophobic phase and is trapped inside the micelle.

Suitable buffer solutions include TAPS, Bicine, Tris, Tricine, TAPSO, HEPES, TES, MOPS, PIPES, and MES. Those of ordinary skill in the art recognize these buffers and know how to make and use them. In some embodiments, the buffer solution keeps the pH of the solution between about 6 to about 8, which is the range most suitable for physiological conditions.

In some embodiments, the buffer solution also contains the building block(s) that can form a hydrogel. In certain embodiments, the hydrogel and its component building block(s) are hydrophilic. In some embodiments, the hydrogel is a polysaccharide. In other embodiments, the hydrogel includes, but is not limited to, poly-(vinyl alcohol), poly-(2-hydroxyethyl methacrylate) (HEMA), poly-(N-isopropyl-2-crylamide), chitosan, hyaluronic acid, gelatin, and sodium alginate. Embodiments include those in which the hydrogel is chitosan or lecithin.

In some embodiments, the mixture of hydrogel and water, and optionally the buffer, is added to cause the micelle inversion. In these embodiments, the hydrogel (e.g., chitosan) remains as a coating over the hydrophilic shell.

Thus, the micelles become trapped within the hydrogel, with the hydrogel completely coating the outer shell of the micelle

s. Chitosan forms an ionic bond with the surfactant, because chitosan is negatively charged, while many surfactants are positively charged. The ionic interaction between the hydrogel and the surfactant provides the user with the opportunity to modify the strength and rigidity of the hydrogel by selecting hydrogels and surfactants that have different charges.

The use of a hydrogel has many advantages. The hydrogel can be prepared in layers of various thickness. The layers allow for easy application of the micelles to the affected areas. The thickness of the hydrogel, which can be controlled by the user, allows for accurate dose control. Micelles that are trapped in the hydrogel are held together by the hydrogel and do not fall apart. Thus, the hydrogel significantly increases the stability of the micelles, which in turn significantly increases the accuracy of the applied dose.

In some embodiments, the drug delivery vehicles disclosed herein are prepared for ophthalmological uses. For some of these embodiments, chitosan was selected as the hydrogel because, in addition to its high tolerability, it can enhance bioadhesion and shows effective corneal penetration.

The drug delivery vehicles disclosed herein are suitable for combination therapy. Other hydrophobic therapeutic agents can be added prior to the inversion step, above, and the hydrophobic agent becomes trapped inside the micelle subsequent to the inversion, just the same as the NO-donor. Hydrophilic drugs can be incorporated either through their own separate encapsulation processes or simply integrating them into the aqueous phase of the hydrogel.

The consistency and the viscosity of the hydrogel can be controlled by adding a copolymer or another agent to the hydrogel. In some embodiments, hyaluronic acid is added to chitosan to improve the consistency of the hydrogel.

In some embodiments, glycerol is added to the hydrogel to result in a softer hydrogel. In certain embodiments, glycerol is added between about 1% to about 5% by weight of the total weight of the mixture.

In some embodiments, the hydrogel solution (e.g., the solution of buffer and hydrogel) is sterilized prior to use. Examples of sterilization, for example by UV irradiation, γ-ray irradiation, autoclave, are well-known in the art. In some embodiments, the micelle solution is sterilized prior to the addition of the hydrogel.

In some embodiments, in addition to the hydrophobic NO-donor trapped inside the lipid nanoparticle, a hydrophilic NO-donor is also added. The hydrophilic NO-donor will be in the hydrogel phase, and therefore, will have a much faster rate of NO release than the NO-donors in the hydrophobic phase inside the micelle. This biphasic NO release allows for a treatment that requires an immediate release formulation of NO (achieved by the hydrophilic NO-donor in the hydrogel phase) combined with an extended-release formulation of NO (achieved by hydrophobic NO-donor in the micelle).

### Crosslinking

In some embodiments, a crosslinking agent is used in combination with the hydrogel. The crosslinked hydrogel is more stable towards degradation under physiological conditions. Also, by varying the extent of crosslinking, a user can vary the viscosity of the hydrogel and prepare a therapeutic composition that best suits the patient's needs.

Various physiologically acceptable crosslinking agents are known in the art. Examples include, but are not limited to, glyoxal, glutaraldehyde, dialdehyde starch, dialdehyde chitosan, the mixture of 1-ethyl-3-(3-dimethylaminopropyl) carbodiimide hydrochloride and N-hydroxysuccinimide (the EDC/NHS mixture), genipin, tannic acid, citric acid, vanillin, and the like. In some embodiments, the crosslinking agent is genipin, citric acid or the EDC/NHS mixture.

In some embodiments, the crosslinking agent is genipin. Genipin not only provides crosslinking at the hydrogel, and therefore affects the viscosity thereof, it can also crosslink the surfactant molecules to the hydrogel, thereby increasing the stability of the drug delivery vehicle and enhancing its adhesion properties.

In some embodiments, the crosslinking agent is the EDC/NHS mixture, combined with citric acid. In this method, the EDC/NHS mixture activates the carboxylic acid group of citric acid and allows the free amines of chitosan to link with the two available carboxylic acid groups of citric acid leading to cross linking. In addition, the citric acid may crosslink chitosan to phosphatidyl choline (PC) in the soy lecithin, leading to increased stabilization of the micelle inside the hydrogel.

In some embodiments, simply heating the mixture, for example in an autoclave during the sterilization step, causes the hydrogel to spontaneously crosslink with itself.

The drug delivery vehicles disclosed herein provide advantages both when the vehicles are in storage and when they are administered to the patient. During storage, the hydrogel-encapsulated lipid nanoparticle prevents the NO-donor from coming into contact with the aqueous environment and any reagents therein that can degrade the NO-donor. This property increases the shelf-life and the stability of the NO-donor.

When the drug delivery vehicles are administered to the patient, the physiological environment of the application site, such as fluids, enzymes, pH, salinity, etc., slowly degrade the hydrogel and allow the lipid nanoparticle to release the NO-donor, and therefore the NO, at the site. Because this process takes time, the release of NO at the site is slow, and the drug delivery vehicle, therefore, can provide an extended-release formulation of NO. The rate of release of the NO can be controlled, as discussed above, by varying the viscosity or the thickness of the hydrogel.

### Packaging

In another aspect, disclosed herein are methods of packaging the drug delivery vehicles disclosed herein.

In some embodiments, the drug delivery vehicles disclosed herein are added to eye drops or eye gels already available on the market, or newly formulated for this application.

In other embodiments, the inside surface of contact lenses is coated with the drug delivery vehicles disclosed herein. When the patient wears the contact lens, the hydrogel comes into contact with the cornea and begins to deliver the NO.

In other embodiments, the drug delivery vehicles disclosed herein are formulated in an injectable formulation, which forms a hydrogel mass within the body that slowly releases the NO.

In other embodiments, the drug delivery vehicles disclosed herein are formulated as infusions, sprays, or patches, depending on the clinical application.

In another aspect, disclosed herein are methods of treating a patient suffering from a disease that can be treated with NO, the method comprising identifying a patient in need thereof, and administering to the patient a drug delivery vehicle as described herein. In some embodiments, the malady is an ophthalmologic disease.

The word “treat,” “treating,” or “treatment” does not denote the total eradication of the diseased state. Any reduction in the disease activity, or any amelioration of the patient's general sense of well-being is considered treatment.

### EXAMPLES

### Example 1: Synthesis of Compound 1

To a 25 mL round bottom flask charged with 5 mL of DMF was added 0.2 g of Ursolic Acid, 0.3 grams of K₂CO₃, and 0.5 grams of 1,2-dibromopentane. The mixture was stirred at 40° C. for 12 hours or until reaction completion was observed using TLC. The mixture was filtered, and the filtrate was reduced in volume using a rotary evaporator. The product (Compound 1) was purified by flash chromatography on silica gel, and was obtained as a yellow oil (0.16, yield=80%).

¹H NMR: (500 MHz, CDCl₃) δ ppm: 5.24 (t, J=3.7 Hz, 1H), 4.00 (ddt, J=10.9, 6.4, 4.6 Hz, 2H), 3.42 (t, J=6.7 Hz, 17H), 1.89 (dq, J=8.1, 6.8 Hz, 19H), 1.77 (td, J=13.6, 4.8 Hz, 1H), 0.85 (d, J=6.4 Hz, 3H).

### Example 2: Synthesis of Compound 2

To a 25 mL round bottom flask charged with 2 mL of acetonitrile was added 0.07 g of Compound 1 and 0.06 g of anhydrous silver nitrate. The mixture was stirred at 85 deg C. for 24 hours or until reaction completion was observed using TLC. The mixture was filtered, and the filtrate was reduced in volume using a rotary evaporator. The product was purified by preparative TLC, and was obtained as a yellow oil (50 mg, yield=74%)

¹H NMR: (500 MHZ, CDCl₃) δ ppm: 5.24 (t, J=3.7 Hz, 1H), 4.47 (t, J=6.5 Hz, 9H), 2.96 (d, J=0.5 Hz, 1H), 2.89 (d, J=0.7 Hz, 1H), 1.20 (d, J=7.0 Hz, 2H), 0.86 (d, J=6.4 Hz, 3H).

### Example 3: Preparation of the Micelle/Hydrogel

EDTA, 40 mg, was dissolved in 100 mL of water. Sodium ascorbate was added until the concentration of sodium ascorbate was under 10% w/v and preferably 6% w/v. Ascorbic acid was added until the pH was between 5 and 5.5. The mixture was heated to between 52-55° C. In some embodiments, but not all, about 0.1-1% glycerol was added as a plasticizer.

A total of 1.5-2 g of chitosan (solid) was added to the mixture to result in a final concentration of 1.5%-2%. The chitosan can be added all at once or stepwise. Either way, it needs to be added slowly. If the chitosan is added stepwise, one half of the total is slowly added to the mixture, followed by the quick addition of 0.15 g of hyaluronic acid, which was then followed by the slow addition of the remaining chitosan. 0.1 gram of sodium citrate is added at this point. The solution can be sonicated to improve clarity. The solution was autoclaved. Genipin, or other crosslinker, was added at this point. The pH was then slowly adjusted to a desirable pH for biological applications (such as 5.5-6.5).

### Example 4: First Case Study: Modeling Structure of PCCM-125

A plasma treated oil (Hadaya, et al., Plasma Medicine, 13(1):65-71 (2023)) underwent an acidified nitration reaction, which resulted in a significant increase (as detected by QUANTOFIX® Nitrate/Nitrite strips, CTL Scientific Supply Corp., Item No. 91313) in the amount of NOx species (nitrite and nitrate) released when the oil was mixed with water. The amount of the NO-donor in the oil was back-approximated based on the final concentration of nitrite/nitrate species.

The NO-donor oil of above was then used to prepare a concentrated emulsion stock. A surfactant system was prepared containing a 1:10:100 ratio of phospholipid (e.g., soy lecithin), TWEEN® 80, and water. The NO-donor oil stock solution was then added to the surfactant system in a 1:100 v/v ratio of oil to surfactant to make the emulsion stock solution.

The core chitosan stock solution was prepared by dissolving chitosan at 2% w/v in 0.008% ascorbic acid under high shear mixing and sonication. Using a combination of glacial acetic acid and sodium bicarbonate, the pH was adjusted to about 5 determined with pH paper. The solution was stirred at high shear and then sonicated overnight. It was then autoclaved. After autoclave, the pH was slowly adjusted to about 6 (on pH paper) using sodium bicarbonate. Genipin was then added at a 1:12 ratio relative to chitosan, and the mixture is allowed to react for about 2 hours. The solution began to turn a green-blue color and ultimately turned a deep green-blue by the next day. Under light microscopy, green spheres are visualized.

The presence of green spheres indicated chitosan was wrapping around micelles, confirming there was crosslinking by genipin surrounding the spheres. The microscopic visualization confirmed that a structure capable of sustained release of its interior was synthesized.

### Example 5: EpiCorneal™ 3D Human Tissue Model

The gel prepared above was used in the EpiCorneal™ 3D human tissue model (MatTek, Ashland, MA, mattek.com).

A similar scratch was made on four pipette tips. Two were used as the control group and two as the test group. The scratches were viewed, and followed throughout the experiment, using confocal microscopy.

Initially a qualitative analysis using ImageJ (a public domain Java image processing program, imagej.net) was conducted to determine if under the experimental conditions, cells would replicate and close the scratch area. The images were first converted to black and white, followed by the application of various masks and filters to better visualize the scratch area. Images were taken at set time points at 24, 48, and 72 hours for both control and test groups.

To our surprise in both test subjects, the wounds closed completely and at a much faster rate than in the controls. The table below shows extent of wound closing as the average for the two samples in each group.

| Time (hrs) | Percent Healing |
|---|---|
| Control Samples | |
| 48 | 36.05 |
| 72 | 62.57 |
| Test Samples | |
| 48 | 100 |

As can be seen, the wound area in the control samples never completely closed after 72 hours. But in both test subjects the wound area entirely closed.

Additionally, the cell morphology appeared more elongated and spindle shaped, which aligns with morphological changes characteristic of enhanced cell migration and possibly proliferation. Cell density appeared qualitatively to have been increased.

### Example 6: Experiment in Lamb Cornea

A standardized mechanical wound model was created using trephination to create wounds ex-vivo in ten lamb corneas. Briefly, an 8 mm circular epithelial wound is created using a surgical trephine, creating the exact same mechanical wound in all 10 corneas. A number 10 surgical blade is then used to scrape away any remaining epithelium to ensure all epithelial tissue in wound area is removed, leading to equal wounds in all 10 corneas.

Three corneas were assigned to the control group and were treated with the PBS buffer only.

Seven corneas were prepared for the test group, and the corneas were treated with a drug delivery vehicle disclosed herein. The test group was further divided into three groups based on the cross-linking nature of the hydrogel. In two samples, the hydrogels were cross-linked with genipin; in two other samples the hydrogels were cross-linked with hyaluronic acid; and in three other samples the hydrogels were not cross-linked. All seven drug delivery vehicles also contained 4% ascorbic acid, and 2% w/v chitosan.

Wounds were evaluated with two standard procedures: gross microscopy and fluorescein staining. Wound healing was observed under the microscope. Similarly, fluorescein staining started at 48 hours and was observed over 4 days. No central wound defect was seen on fluorescein staining on any of the experimental samples. Conversely, a central defect was persistent over all 4 days for the control samples.

1. A drug delivery vehicle composition, comprising lipid nanoparticle-forming molecules, a nitric oxide donor (NO-donor) molecule, and a hydrogel.

2. The composition of claim 1, wherein the NO-donor molecule is a direct donor or an indirect donor.

3. The composition of claim 1, wherein the NO-donor molecule is prepared from an inert triterpenoid compound.

4. The composition of claim 1, wherein the NO-donor molecule is an organic nitrate ester made by the process of 1) reacting the inert triterpenoid compound with a haloalkane in a polar solvent, and optionally in the presence of a mild base, and 2) reacting the product of step 1 with silver nitrate in a polar solvent.

5. The composition of claim 4, wherein the inert triterpenoid compound is selected from the group consisting of ursolic acid, 23-hydroxy ursolic acid, oleanolic acid, uvaol, -amyrin, amyrin, asiatic acid, erythrodiol, hederagenin, madecassic acid, corosolic acid, betulin, and bardoxolone.

6. The composition of claim 1, wherein the NO-donor molecule has the structure:

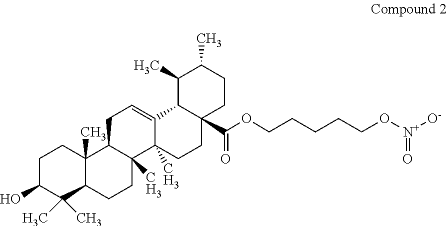 [SMILES: C[C@H]1[C@H](C)CC[C@]2(C(=O)OCCCCCO[N+](=O)[O-])CC[C@]3(C)C(=CC[C@@H]4[C@@]5(C)CC[C@H](O)C(C)(C)[C@@H]5CC[C@]43C)[C@H]12]

7. The composition of claim 1, wherein the lipid nanoparticle-forming molecule is amphiphilic.

8. The composition of claim 1, wherein the lipid nanoparticle-forming molecule is a lipid selected from the group consisting of free fatty acids, glycerolipids, phospholipids, glycerophospholipids, sphingolipids, saccharolipids, polyketides, sterol lipids, and prenol lipids.

9. The composition of claim 1, wherein the lipid nanoparticle-forming molecule has an alkyl chain comprising between about 5 to about 30 carbon atoms.

10. The composition of claim 1, wherein the lipid nanoparticle-forming molecule is polyoxyethylene (80) sorbitan monooleate or lecithin.

11. The composition of claim 1, wherein the lipid nanoparticle is a micelle or a microemulsion.

12. The composition of claim 1, further comprising a surfactant.

13. The composition of claim 12, wherein the surfactant is an oleate-derived surfactant or a laurate-derived surfactant.

14. The composition of claim 1, wherein the hydrogel is a polysaccharide.

15. The composition of claim 1, wherein the hydrogel comprises poly-(vinyl alcohol), poly-(2-hydroxyethyl methacrylate) (HEMA), poly-(N-isopropyl-2-crylamide), chitosan, hyaluronic acid, gelatin, and sodium alginate.

16. The composition of claim 1, wherein the hydrogel further comprises a crosslinking agent.

17. The composition of claim 16, wherein the crosslinking agent is selected from the group consisting of glyoxal, glutaraldehyde, dialdehyde starch, dialdehyde chitosan, the mixture of 1-ethyl-3-(3-dimethylaminopropyl) carbodiimide hydrochloride and N-hydroxysuccinimide (the EDC/NHS mixture), genipin, tannic acid, citric acid, and vanillin.

18. A method of making a drug delivery vehicle composition, wherein the composition comprises lipid nanoparticle-forming molecules, a nitric oxide donor (NO-donor) molecule, and a hydrogel, the method comprising the steps of:
mixing the lipid nanoparticle-forming molecules and the NO-donor in a hydrophobic solvent to form a first mixture; and
adding a mixture of hydrogel and water, and optionally a buffer solution, to the first mixture to form the drug delivery vehicle composition.

19. The method of claim 18, wherein the hydrophobic solvent is dimethylformamide.

20. The method of claim 18, wherein the buffer solution is selected from the group consisting of TAPS, Bicine, Tris, Tricine, TAPSO, HEPES, TES, MOPS, PIPES, and MES.

21. A method of treating a patient suffering from a disease that can be treated with nitric oxide, the method comprising identifying a patient in need thereof, and administering to the patient a drug delivery vehicle of claim 1.

22. The method of claim 21, wherein the disease is an ophthalmologic disease.
